(19) United States
(12) Patent Application Publication
(10) Pub. No.: US 2019/0313678 A1
(43) Pub. Date: Oct. 17, 2019

(54) SUGAR-DIPEPTIDE CONJUGATES AS FLAVOR MOLECULES

(71) Applicant: NESTEC S.A., Vevey (CH)

(72) Inventors: Candice Marie Smarrito-Menozzi, Belmont-sur-Lausanne (CH); Florian Viton, Lausanne 26 (CH); Maria Eugenia Barcos, Epalinges (CH); Sonia Manganiello, Neuchatel (CH)

(21) Appl. No.: 16/314,724

(22) PCT Filed: Jul. 6, 2017
(86) PCT No.: PCT/EP2017/066882

§ 371 Date: Jan. 2, 2019

(30) Foreign Application Priority Data
Jul. 7, 2016 (EP) 16178420.2

(51) Int. Cl. A23L 27/20; C07H 15/12; C07K 5/02; A23L 13/40; A23L 23/10; A23L 27/21; A23L 27/26; A23L 27/00

(52) U.S. Cl. CPC A23L 27/2052; C07H 15/12; C07K 5/0202; A23L 13/428; A23V 2002/00; A23L 27/21; A23L 27/26; A23L 27/88; A23L 23/10

(57) ABSTRACT

The present invention relates to compounds and compositions for use in enhancing flavor and/or saltiness of a food product. Particularly, the present invention relates to compounds of the general formula I) and compositions comprising them.

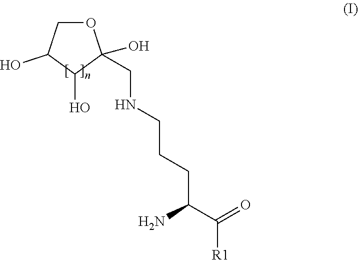

The present invention relates to compounds and compositions for use in enhancing flavor and/or saltiness of food products.

Many foods that are consumed today are rich in umami and/or meaty taste and flavor. Umami or meaty taste of a food product can for example be achieved or enhanced by adding separately monosodium glutamate (MSG) and/or the ribonucleotides GMP and IMP into those culinary recipes. Many such taste enhancers are available today and are used for various different culinary applications and in various different forms such as pastes, powders, liquids, compressed cubes or granules.

The addition of culinary additives helps to provide deliciousness and to enhance taste and flavor properties of food products. And indeed, all around the world taste and flavor is perceived as one of the key attributes of a high quality meal. Hence, a lot of research efforts goes into the identification and analysis of new molecules providing deliciousness, and enhanced taste and flavor properties of foods.

For example, in a recent scientific publication from A. Dunkel and T. Hofmann (Dunkel and Hofmann, 2009, J. Agric. Food Chem. 2009, 57, 9867-9877), sensory-directed fractionation of a freshly prepared double-boiled chicken soup led to the identification of the β-alanyl dipeptides L-anserine, L-carnosine and β-alanylglycine as contributors to the thick-sour and white-meaty orosensation. Quantitative analysis, followed by taste recombination and omission experiments, revealed for the first time that, when present together with L-glutamic acid and sodium and/or potassium ions, subthreshold concentrations of these three β-alanyl peptides enhance the typical thick-sour orosensation and white-meaty character known for poultry meat. This is a first step in finding new compounds which are able to impart flavour richness and enhance the umami taste effect of glutamate.

T. Sonntag et al., J. Agric. Food Chem. 2010, 58, 6341-6350, described sensory-guided fractionation of stewed beef juice which led to the identification of several taste active dipeptides and creatinine derivatives. Quantitative analysis revealed that subthreshold concentrations of such taste modulator molecules enhance the typical thick-sour and mouth-drying orosensation imparted by stewed beef juice.

J. P. Ley, Chem. Percept. 2008, 1:58-77, reviewed bitter taste masking molecules potentially useful in food and beverage products. Particularly, Ley described ornithine and ornithine derivatives such as L-ornithyl-β-alanine or L-ornithinyltaurine as having bitter masking effects against potassium salts. L-Ornithine was also described as an off-taste inhibitor in combination with bitter branched chain amino acids.

M. Tamura et al., Agric. Biol. Chem., 1989, 53 (2), 319-325, described the relationship between taste and primary structure of a delicious peptide from beef soup that produced a salty and umami taste as well as delicious taste. They referred to earlier work where they for example synthesized ornithyl-taurine and found that it produced a salty taste. Similar taste effect was discovered with similar other dipeptides.

The object of the present invention is to improve the state of the art and to provide an alternative or improved solution to the prior art to overcome at least some of the inconveniences described above. Particularly, the object of the present invention is to provide an alternative or improved solution for enhancing the taste and flavour of food products. Particularly, the object of the present invention is to improve the taste, as for example the delicious, umami and/or salty taste, of a food product. The object or the present invention is also to improve the flavour of food products as for example meat flavour, bread flavour, and particularly grilled meat or bread flavour.

The object of the present invention is achieved by the subject matter of the independent claims. The dependent claims further develop the idea of the present invention.

Accordingly, the present invention provides in a first aspect a compound of the general formula I,

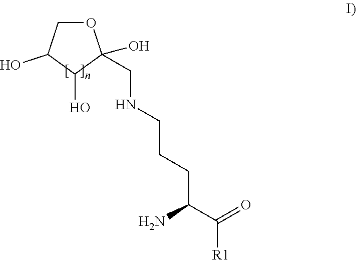

wherein R1 is hydroxyl (—OH) or an acid comprising an amino group, and wherein n is equal 1 or 2; or a salt of said compound.

In a second aspect, the invention relates to a composition comprising said compound of the general formula I) in an amount of at least 0.25 mg/g, preferably of at least 0.5 mg/g, 1.0 mg/g, 1.5 mg/g, 2.5 mg/g, or 5 mg/g of the total composition.

Further aspects of the present invention relate to a use of said compound for enhancing the flavor and/or saltiness of a food product.

A still further aspect of the present invention is a method for enhancing the flavor and/or saltiness of a culinary food product, comprising the step of adding said compound or the composition comprising said compound to a food product.

The inventors surprisingly found that some sugar conjugates of ornithine and ornithine-dipeptides have a much stronger flavor enhancing effect than their corresponding aglycones. In fact, these sugar conjugates enhance the grilled meat and bread flavor and salt taste perception at much lower threshold levels than their corresponding aglycones. They also enhance the persistency of those flavors and tastes in the mouth, while at the same the concentration their corresponding aglycones does not impact the flavors per se. Such sugar conjugate molecules are typically generated in-situ during thermal processing of food raw materials by condensation of a reducing sugar with e.g. ornithine or an ornithine-dipeptide such as e.g. L-ornithinyl-β-alanine or L-ornithinyl taurine. The corresponding aglycones, i.e. the ornithine and ornithine-dipeptides, have been identified and described for example by Sonntag et al., 2010, J. Agric. Food Chem. 58, 6341-6350; by J. P. Ley, Chem. Percept. 2008, 1:58-77; and by M. Tamura et al., Agric. Biol. Chem., 1989, 53 (2), 319-325.

However, the taste properties of these ornithine derivatives differ from the ones of their corresponding sugar conjugates. Evidence thereof is provided in the Example section below. Therefore, the molecules described in the present invention are more potent flavor and salt taste enhancers than the known ornithine and ornithine-dipeptides. They allow further reducing the amounts and uses of for example mono-sodium glutamate (MSG), of ribonucleotides such as IMP and GMP, and of regular kitchen salt in culinary food products and applications, without compromising flavor richness, deliciousness and salt perception of said products. They also allow generating savory food concentrates which have much less or no MSG, ribonucleotides and/or salt, and which still provide a strong and typical delicious, meaty, salt tasting effect if applied to a food product. It even allows generating such savory food concentrates which are much stronger and more concentrated in providing a grilled meaty or salty taste to a food product upon application.

### BRIEF DESCRIPTION OF THE DRAWINGS

FIG. 1: Sensory evaluation of chicken soups spiked with 2 g/L Fru-orn-βala (a) and orn-ala (b). Sensory scores of the taste/flavor attributes are shown as differences (positive and negative) versus the corresponding score from a reference chicken soup sample without the orn compounds. *) indicates statistic significant differences.

FIG. 2: Sensory evaluation of chicken soups spiked with Fru-Orn at 2 g/L (a) and 0.25 g/L (b) concentrations. Sensory scores of the taste/flavor attributes are shown as differences (positive and negative) versus the corresponding score from a reference chicken soup sample without the orn compound. *) indicates statistic significant differences.

### DETAILED DESCRIPTION OF THE INVENTION

The present invention pertains to a compound of the general formula I), wherein R1 is hydroxyl (—OH) or an acid comprising an amino group, and wherein n is equal 1 or 2; or a salt of said compound. Particularly, the acid comprising the amino group can be selected from the group consisting of alanine, taurine, aspartic acid and glutamic acid.

When n is equal 1, the sugar moiety of the compound of the general formula I) is preferably selected from xylose or ribose. The compound is then preferably a derivative of xylose or ribose.

When n is equal 2, the sugar moiety of the compound of the general formula I) is preferably a glucose. The compound is then preferably a derivative of glucose.

Preferably, the compound of the present invention is selected from the group consisting of 1-deoxy-D-fructosyl-N-ornithine, 1-deoxy-D-fructosyl-N-ornithyl-β-alanine, 1-deoxy-2-pentulofuranos-1-yl-ornithine, and 1-deoxy-2-pentulofuranos-1-yl-ornithyl-β-alanine.

A second aspect of the invention relates to a composition comprising said compound of the general formula I) in an amount of at least 0.25 mg/g, at least 0.50 mg/g, at least 0.75 mg/g, at least 1.0 mg/g, at least 1.5 mg/g, at least 1.7 mg/g, at least 2 mg/g, at least 2.5 mg/g, at least 3 mg/g, at least 3.5 mg/g, or at least 5 mg/g of the total composition.

In one embodiment of the present invention, the composition is in the form of an extract from a plant, fungus and/or meat material. Preferably, the composition is in the form of an extract, for example from plant, fungus and/or meat material, where the compound of the present invention has been enriched. An advantage thereby is that the composition is of natural origin and does not contain any chemically synthesized compounds.

In another embodiment, the composition of the present invention is the result of a flavor reaction. The term “flavor reaction” refers herein to a chemical reaction occurring between at least one reducing sugar and at least one amino acid or protein. Typically, this chemical reaction occurs during a heating process and is typically also referred to as Maillard reaction. In one example, the flavor reaction is a Maillard reaction.

In a preferred embodiment, the composition of the present invention is food grade. Under “food grade” the inventors mean that the composition is suitable for human consumption, for example directly, in concentrated form, and/or when used diluted in a food product.

For example, the composition of the present invention is selected from the group consisting of a culinary seasoning product, a cooking aid, a sauce or soup concentrate, a dry or wet pet-food product.

Further aspects of the present invention relate to a use of said compound for enhancing the flavor and/or taste of a food product. Such a food product may be a ready-to-eat food product. It may also be a flavor concentrate used for seasoning a still further other food product. Advantageously, the compound of the present invention may be used for being added to a seasoning, a cooking aid or a food concentrate product. Thereby the strength of providing e.g. a meaty or a salty taste to a still further food product is improved in such a seasoning, cooking aid or food concentrate product.

Particularly, the present invention also relates to the use of the compounds for enhancing the grilled meat or bread flavor of a food product.

Furthermore, the invention also relates to the use of the compounds of the present invention for enhancing the saltiness of a food product. Particularly, this use would allow to either increase the perceived saltiness of a food product without actually increasing the salt or sodium level of said food product, or to decrease the amount of salt or sodium used in a food product with maintaining the actual perceived saltiness of said product. Advantageously thereby the amount of salt and sodium consumed by consumers with such a product today could be significantly reduced.

Further aspects of the present invention also relate to a use of a composition comprising said compound in an amount of at least 0.25 mg/g, at least 0.50 mg/g, at least 0.75 mg/g, at least 1.0 mg/g, at least 1.5 mg/g, at least 1.7 mg/g, at least 2 mg/g, at least 2.5 mg/g, at least 3 mg/g, at least 3.5 mg/g, or at least 5 mg/g of the total composition, for enhancing the flavor and/or saltiness of a food product. Advantageously, such a food product may be a ready-to-eat food product.

A still further aspect of the present invention is a method for enhancing the flavor and/or saltiness of a culinary food product, comprising the step of adding said compound or the composition comprising said compound to a food product. The food product can be a ready-to-eat food product or a flavor concentrate.

Those skilled in the art will understand that they can freely combine all features of the present invention disclosed herein. In particular, features described for the products of the present invention may be combined with the uses and method of the present invention, and vice versa. Further, features described for different embodiments of the present invention may be combined. Further advantages and features of the present invention are apparent from the figures and examples.

### Example 1: Synthesis or Preparation of 1-deoxy-D-fructosyl-N-ornithyl-β-alanine (Fru-Orn-βala)

### Step-1: Synthesis of benzyl (S)-3-(2-((((9H-fluoren-9-yl)methoxy)carbonyl)amino)-5-((tert-butoxycarbonyl)amino) pentanamido)propanoate I

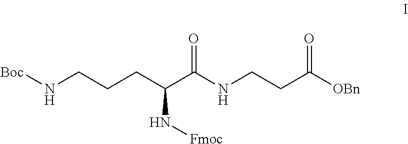

| | | | | Eq. | |
|---|---|---|---|---|---|
| Compound No./Reagents | Qty. | | mmol | Ratio | Yield |
| 2-((((9H-fluoren-9- | 15.0 | g | 33.039 | 1.0 | 15.0 g |
| yl)methoxy)carbonyl)amino)- | | | | | (73.85%) |
| 5-((tert-butoxycarbonyl) | | | | | |
| amino)pentanoic acid | | | | | |
| Benzyl 3-aminopropanoate | 20.69 | g | 115.638 | 3.5 | |
| 1-Ethyl-3-(3- | 8.20 | g | 42.951 | 1.3 | |
| dimethylaminopropyl)carbodi- | | | | | |
| imide hydrochloride | | | | | |
| (EDC•HCl) | | | | | |
| Hydroxybenzotriazole | 2.23 | g | 16.519 | 0.5 | |
| (HOBT) | | | | | |
| Dichloromethane | 600 | mL | — | 40 vol | |

2-((((9H-fluoren-9-yl)methoxy)carbonyl)amino)-5-((tert-butoxycarbonyl)amino)pentanoic acid (15.0 g, 33.039 mmol, 1.0 eq.) was dissolved in dichloromethane (600 mL) followed by addition of benzyl 3-aminopropanoate (20.69 g, 115.638 mmol, 3.5 eq), EDC.HCl (8.20 g, 42.951 mmol, 1.3 eq.), HOBT (2.23 g, 16.519 mmol, 0.5 eq.) at room temperature. The reaction was stirred at room temperature for 5 hours. The mixture was then diluted with dichloromethane (300 mL) and washed with a saturated bicarbonate solution (300 mL). The organic layer was dried over Na₂SO₄ and concentrated under reduced pressure to give a crude that was purified by column chromatography using neutral silica gel of 60-120 mesh size. 0-60% ethyl acetate was used as gradient in hexane for elution. After evaporation of the solvent under reduced pressure, 15.0 g compound I (73.85%) were obtained.

### Step-2: Synthesis of benzyl (S)-3-(2-((((9H-fluoren-9-yl)methoxy)carbonyl)amino)-5-aminopentanamido)propanoate II

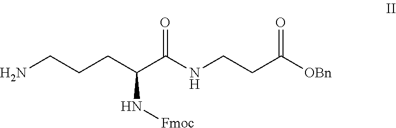

| | | | | Eq. | |
|---|---|---|---|---|---|
| Compound No./Reagents | Qty. | | mmol | Ratio | Yield |
| Compound I | 15.0 | g | 24.390 | 1.0 | 11.9 g |
| HCl in 1,4-dioxane (4M) | 20 | mL | — | — | (94.74%) |
| Dichloromethane | 300 | mL | — | 20 vol. | |

Compound I (15.0 g, 24.390 mmol, 1.0 eq.) was dissolved in dichloromethane (300 mL) and HCl in 1,4-dioxane (4 M) was slowly added at 0° C. The resulting mixture was stirred at room temperature for 4 hours. The reaction mass was then concentrated under reduced pressure to give 11.9 g of compound II (94.74%).

### Step-3: Synthesis of 3-((2S)-2-((((9H-fluoren-9-yl) methoxy) carbonyl)amino)-5-(((2,3,4,5-tetrahydroxytetrahydro-2H-pyran-2-yl)methyl)amino)pentanamido)propanoic acid III

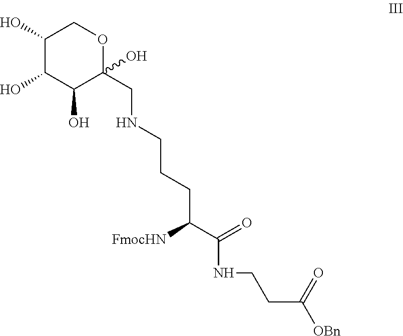

| | | | | Eq. | |
|---|---|---|---|---|---|
| Compound No./Reagents | Qty. | | mmol | Ratio | |
| Compound III | 11.8 | g | 22.912 | 1.0 | Yield = |
| D-Glucose | 11.54 | g | 64.155 | 2.8 | 13.4 g |
| Sodium bisulphite | 0.66 | g | 6.415 | 0.28 | (100%) |
| Glycerol | 20 | mL | — | — | |
| Acetic acid | 7 | mL | — | — | |
| Methanol | 80 | mL | — | — | |
| Water | 80 | mL | | | |

D-Glucose (11.54 g, 64.155 mmol, 2.8 eq.) and sodium bisulphite (0.66 g, 6.415 mmol, 0.28 eq.) were taken up in a mixture of methanol (80 mL) and glycerol (20 mL). The reaction mixture was refluxed for 30 min at 80° C. followed by the addition of compound II (11.8 g, 22.912 mmol, 1.0 eq.) and acetic acid (7 mL). The reaction mass was heated at 100° C. for 5 hours and concentrated under reduced pressure to give 13.4 g of final compound III which was used without any further purification.

### Step-4: Synthesis of Fru-orn-βala

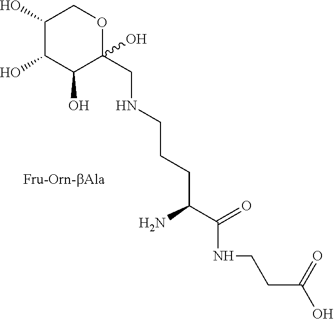 [SMILES: N[C@@H](CCCNCC1(O)OC[C@@H](O)[C@@H](O)[C@@H]1O)C(=O)NCCC(=O)O]

| | | | | Eq. | |
|---|---|---|---|---|---|
| Compound No./Reagents | Qty. | | mmol | Ratio | Yield |
| Compound III | 13.4 | g | 22.827 | 1.0 | 4.2 g |
| 10% Pd on Carbon (50% | 4.0 | g | — | — | (50.42%) |
| Moisture) | | | | | |
| MeOH | 400 | mL | — | 40 vol. | |

Compound III (13.4 g, 22.827 mmol, 1.0 eq.) was dissolved in MeOH (400 mL) and 10% Pd on Carbon (50% moisture) was slowly added. The resulting suspension was stirred at room temperature for 6 hours under H₂ atmosphere. After completion, the reaction mass was filtered through Celite, washed with water and concentrated under reduced pressure to give 8.0 g of crude which was then poured onto a packed-column (Amberlite IRN-77 ion exchange resin, 100 g) and eluted with 0.2% NH₃ in water. The collected fractions were evaporated under reduced pressure to obtain 4.2 g of Fru-Orn-βala (50.42%).

¹H NMR spectrum in D₂O: 1.582-1.591 (d, 4H), 2.277-2.310 (t, 2H), 2.889-2.924 (m, 2H), 3.115-3.136 (m, 2H), 3.232-3.261 (m, 1H), 3.359-3.376 (m, 2H), 3.604-3.629 (m, 2H), 3.749-3.774 (m, 1H), 3.877-3.907 (m, 2H).

LC-MS was carried out using Sunfire C18 (250×4.6 mm, 5 μm). The column flow was 0.3 mL/min and solvents used were 0.1% TFA in water (A) and MeOH (B). The elution method: isocratic 95% A and 5% B.

| | Molecular ion peak | Wavelength | Retention time |
|---|---|---|---|
| Fru-Orn-βAla | 366.25 [M + H]⁺ | 202 nm | 10.082 |

### Example 2: Synthesis or Preparation of 1-deoxy-D-fructosyl-N-ornithine (Fru-Orn)

### Step-1: Synthesis of benzyl 5-amino-2 (((benzyloxy)carbonyl) amino) pentanoate IV

| | | | | |
|---|---|---|---|---|
| | | | Eq. | |
| Reagents | Qty. | mmol | Ratio | Yield |

| | | | | | |
|---|---|---|---|---|---|
| Z-Orn-OH | 20.0 | g | 75.187 | 1.0 | 17.0 g |
| PTSA•H₂O | 14.28 | g | 75.187 | 1.0 | (63.5%) |
| Benzyl alcohol | 24.36 | g | 225.563 | 3.0 | |
| Toluene | 400 | mL | — | 20 vol. | |

To a stirred solution of Z-Orn-OH (20.0 g, 75.187 mmol, 1.0 eq.) in toluene, PTSA.H₂O (14.28 g, 75.187 mmol, 1.0 eq.) and benzyl alcohol (24.36 g, 225.563 mmol, 3.0 eq.) were added at room temperature and the mixture was stirred overnight at 120° C. while water was collected using a Dean Stark separator. After completion, the reaction mixture was cooled down to 0° C. and diluted with diethyl ether. The product was filtered and dried under reduced pressure to give 17.0 g compound IV (63.5%).

### Step-2: Synthesis of benzyl 2-(((benzyloxy)carbonyl)amino)-5-(((2,3,4,5-tetrahydroxy-tetrahydro-2H-pyran-2-yl)methyl)amino)pentanoate V

| | | | | |
|---|---|---|---|---|
| | | | Eq. | |
| Reagents. | Qty. | mmol | Ratio | Yield |

| | | | | | |
|---|---|---|---|---|---|
| Glucose | 22.65 | g | 125.842 | 2.8 | 15.8 g |
| Compound IV | 16.0 | g | 44.943 | 1.0 | (67.86%) |
| Sodium bisulphite | 1.30 | g | 12.584 | 0.28 | |
| Glycerol | 2 0 | mL | — | — | |
| Acetic acid | 5.1 | mL | — | — | |
| Methanol | 60 | mL | — | — | |
| Water | 60 | mL | | | |

D-Glucose (22.65 g, 125.842 mmol, 2.8 eq.) and sodium bisulphite (1.3 g, 12.584 mmol, 0.28 eq.) were taken up in a mixture of methanol (60 mL) and glycerol (20 mL). The reaction mixture was refluxed for 30 min at 80° C. followed by the addition of Compound IV (16.0 g, 44.943 mmol, 1.0 eq.) and acetic acid (5.1 mL). The reaction mass was heated at 80° C. for 3 hours, cooled down and diluted with water (60 mL). The mixture was then poured onto a packed-column (Amberlite IRN-77 ion exchange resin, 160 g). The crude was eluted with water and the collected fractions were evaporated under reduced pressure to obtain 15.8 g pure compound V (67.86%).

### Step-3: Synthesis of Fru-Orn

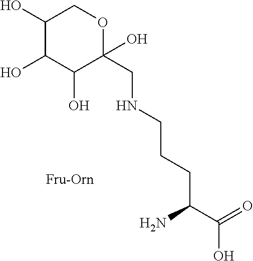 [SMILES: N[C@@H](CCCNCC1(O)OCC(O)C(O)C1O)C(=O)O]

| | | | | Eq. | |
|---|---|---|---|---|---|
| Reagents | Qty. | | mmol | Ratio | Yield |
| Compound V | 15.8 | g | 30.501 | 1.0 | 4.8 g |
| 10% Pd on Carbon (50% | 5.0 | g | — | — | (53.57%) |
| Moisture) | | | | | |
| MeOH | 400 | mL | | | |

Compound V (15.8 g, 30.501 mmol, 1.0 eq.) was dissolved in MeOH (400 mL) and 10% Pd on Carbon (50% moisture) was slowly added. The resulting mixture was stirred at room temperature overnight under H₂ atmosphere. After completion, the reaction mass was filtered through Celite, washed with water and concentrated under reduced pressure. The crude was then poured onto a packed-column (Amberlite IRN-77 ion exchange resin, 100 g) and eluted with 0.5% NH₃ in water. The collected fractions were evaporated under reduced pressure to obtain 4.8 g Fru-Orn (53.57%).

¹H NMR spectrum in D₂O: 1.679-1.814 (m, 4H), 2.920-2.957 (t, 2H), 3.055-3.072 (d, 2H), 3.197-3.242 (t, 2H), 3.552-3.672 (m, 3H), 3.759-3.791 (m, 1H), 3.876-3.905 (d, 2H).

LC-MS was carried out using Sunfire C18 (250×4.6 mm, 5 μm). The column flow was 0.5 mL/min and solvent was 20 mM ammonium acetate+0.1% acetic acid in water. Elution method: isocratic (100% A).

| | Molecular ion peak | Wavelength | Retention time |
|---|---|---|---|
| Fru-Orn | 295.10 [M + H]⁺ | 236 nm | 4.710 |

### Example 3: Synthesis or Preparation of 1-deoxy-2-pentulofuranos-1-yl-ornithine (Rib-Orn)

### Step-1: Synthesis of (2S)-2-(((benzyloxy)carbonyl)amino)-5-((((3R,4R)-2,3,4-trihydroxytetrahydrofuran-2-yl)methyl)amino)pentanoic acid

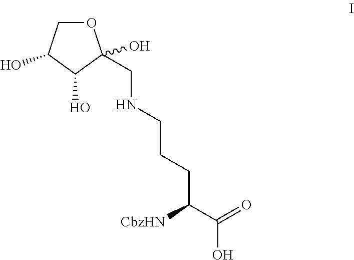 [SMILES: O=C(N[C@@H](CCCNCC1(O)OC[C@@H](O)[C@H]1O)C(=O)O)OCc1ccccc1]

| | | | | Eq. | |
|---|---|---|---|---|---|
| Reagents | Qty. | | mmol | Ratio | Yield |
| D-Ribose | 22.55 | g | 150.375 | 4 | 10.0 g |
| Carboxybenzylornithine | 10.0 | g | 35.593 | 1.0 | (66.66%) |
| (Z-Orn-OH) | | | | | |
| Acetic acid | 0.6 | mL | — | — | |
| Methanol | 800 | mL | — | — | |

D-Ribose (22.55 g, 150.375 mmol, 4.0 eq.) was suspended in Methanol (800 mL). The reaction mixture was refluxed for 60 min at 90° C. followed by the addition of Z-Orn-OH (10.0 g, 35.593 mmol, 1.0 eq.) and ACOH (0.6 mL). The reaction mass was heated at 90° C. for further 1 hr. After completion, the reaction mass was freeze-dried to give a final crude compound which was purified by precipitation in MeOH:ACN (1:5). Solid product was freeze-dried to give 10.0 g of pure compound I (66.66%).

### Step-2: Synthesis of (2S)-2-amino-5-((((3R,4R)-2,3,4-trihydroxytetrahydrofuran-2-yl)methyl)amino)pentanoic acid

| | | | | Eq. | |
|---|---|---|---|---|---|
| Reagents | Qty. | | mmol | Ratio | Yield |
| (2S)-2- | 10.0 | g | 25.062 | 1.0 | 4.8 g |
| (((benzyloxy)carbonyl)amino)-5- | | | | | (69.69%) |
| ((((3R,4R)-2,3,4- | | | | | |
| trihydroxytetrahydrofuran-2- | | | | | |
| yl)methyl)amino)pentanoic acid | | | | | |
| (Compound-I) | | | | | |
| 10% Pd on Carbon (50% | 4.0 | g | | | |
| Moisture) | | | | | |
| Methanol | 60 0 | mL | — | — | |

(2S)-2-(((benzyloxy)carbonyl)amino)-5-((((3R,4R)-2,3,4-trihydroxytetrahydrofuran-2-yl)methyl)amino)pentanoic acid (Compound-I) (10.0 g, 25.062 mmol, 1.0 eq.) was dissolved in MeOH (600 mL) and 10% PD on Carbon (50% moisture) was slowly added. The resulting mixture was stirred at room temperature for 2 hr under H₂ atmosphere. After completion, the reaction mass was filtered through Celite and washed with water. It was freeze-dried to give 4.8 g of pure compound Rib-Orn (Yield: 69.69%).

¹H NMR spectrum in D₂O: 1.700-1.816 (m, 4H), 3.042-3.188 (m, 2H), 3.213-3.245 (m, 1H), 3.543-3.552 (m, 1H), 3.746-3.964 (m, 3H), 4.037-4.351 (m, 2H).

LC-MS was carried out using X-Bridge C18 column (250×4.6 mm, 5 μm). The column flow was 0.5 mL/min and the solvent was 10 mM ammonium acetate+0.3% NH₃ in water. Elution method: isocratic (100% A).

| | Molecular ion peak | Wavelength | Retention time |
|---|---|---|---|
| Rib-Orn | 265.28 [M + H]⁺ | 202 nm | 6.54 |

### Example 4: Sensory Evaluation of the Compounds in Water

The compounds ornithine, ornithyl-β-alanine, 1-deoxy-D-fructosyl-N-ornithine, 1-deoxy-D-fructosyl-N-ornithyl-β-alanine and 1-deoxy-2-pentulofuranos-1-yl-Ornithine (Rib-Orn) were each dissolved and diluted in water in a final concentration of 2 g/L. The solutions were then evaluated by 12 panelists, which were previously screened and selected for their sensory abilities. The results of the sensory evaluation can be summarized as follows: While the aqueous solution with the L-ornithine was reported in the literature as being sweet, the aqueous solution with the ornithyl-β-alanine dipeptide was perceived as astringent; the aqueous solution with the 1-deoxy-D-fructosyl-N-ornithine was described as salty, and the aqueous solution with Rib-Orn was perceived astringent, sour, metallic and slightly sweet.

### Example 5: Sensory Evaluation of the Compounds in a Chicken Soup Base

### Sample Preparation:

Chicken soups were prepared by dissolving 6 g chicken base powder (detailed recipe shown in Table 1), 1 g monosodium glutamate and 1 g of sodium chloride in 500 mL hot water. 1-Deoxy-D-fructosyl-N-ornithyl-β-alanine, N-ornithyl-β-alanine, 1-deoxy-D-fructosyl-N-ornithine or alternatively 1-deoxy-2-pentulofuranos-1-yl-Ornithine (Rib-Orn) were then added separately at 2 and 0.25 g/L final concentrations.

TABLE 1
Composition of the chicken base powder

| Ingredient | Quantity (wt %) |
|---|---|
| Chicken Meat powder | 30 |
| Starch | 1.52 |
| Flavors | 2.58 |
| Celery powder | 0.50 |
| Garlic powder | 0.90 |
| Chicken fat | 8.00 |
| Maltodextrine | 56.50 |
| Total | 100 |

### Sensory Protocol:

The sensory evaluation was carried out by 12 panelists, previously screened for their sensory abilities. The panelists assessed a maximum of 6 samples per session. They had Vittel water and crackers as mouth cleansers. In all the cases, the panelists were instructed to evaluate the samples for the following attributes: overall flavor persistency, meaty, grilled/popcorn-like, bread-like, sweet, bitter, and salty. The samples were coded with random 3-digit numbers according to a balanced presentation design, heated at approximately 65° C. and then presented in 40 ml brown plastic containers and under red light to minimize appearance bias (the serving was approximately 25 ml per sample).

### Statistical Analysis of the Results:

The following statistical tests were used to analyze the sensory raw data: First, the Analysis of Variance (ANOVA) with two factors, products (fixed factor) and subject (random factor) was computed in order to determine if there was any difference among samples. The limit of significance (alpha risk) was set at 5%. Then, the Least Significant Difference (LSD) multiple paired comparison test was performed when a significant difference was detected with the ANOVA, in order to determine which pairs of samples were significantly different. The limit of significance (alpha risk) was set at 5%.

### Results of the Sensory Evaluation

Comparison Between Reference Soup (REF) and the Soup Containing the Dipeptide N-Ornithyl-β-Alanine (orn-ala):

Despite that fact that the dipeptide orn-ala was perceived in the evaluation in aqueous solution as astringent, no statistically significant differences could be observed when comparing the diluted chicken soup base with or without the orn-ala dipeptide at a concentration of 2 g/L. This was true for all the above listed sensory attributes, i.e. overall flavor persistency, meaty, grilled/popcorn-like, bread-like, sweet, bitter, and salty. This means that at a low concentration of 2 g/L, the orn-ala dipeptide does not impact the flavor profile of a simple chicken soup base.

Comparison Between the Soup Bases Containing the Dipeptide N-ornithyl-β-alanine (orn-ala) and the Sugar Conjugate 1-Deoxy-D-fructosyl-N-ornithyl-β-alanine (Fru-Orn-βala):

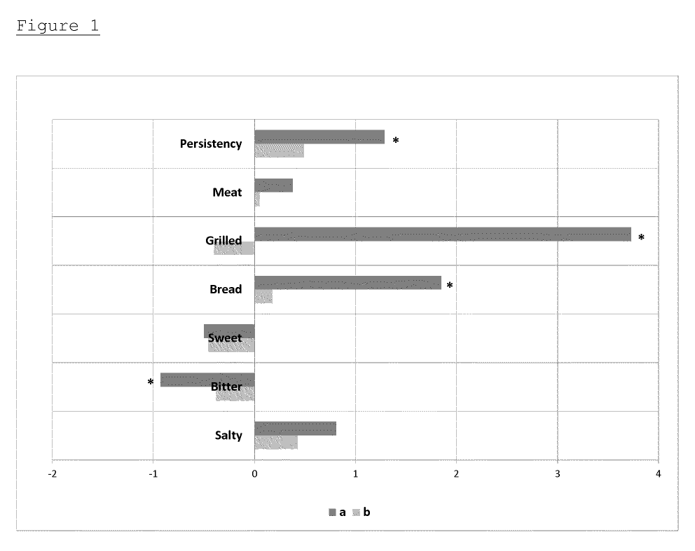

When comparing the sensory profile of the soup comprising the dipeptide orn-ala with the soup comprising the Fru-Orn-βala at each same concentrations of the ornithine compounds of 2 g/Liter, bitterness, bread, grilled aroma as well as the overall flavor persistency were significantly different. In fact, the sugar conjugate Fru-Orn-βala enhanced significantly the grilled and bread attributes, while at the same time it significantly decreasing the perceived bitterness attribute of the chicken soup. The result is shown in FIG. 1.

In conclusion, addition of the sugar conjugate Fru-Orn-βala positively modulates the flavor profile of the chicken soup and increases the desired grilled meat and bread flavor note, while reducing overall bitterness, if directly compared to the impact of its respective control dipeptide orn-ala.

Chicken Soups with Sugar Conjugate of Ornithine at Different Concentrations:

When the glucose conjugate of ornithine, i.e. 1-deoxy-D-fructosyl-N-ornithine, was added to the chicken soup base at 0.25 g/L and its flavor impact tested, the desired bread flavors were significantly perceived in comparison to the reference soup base. However, it was at the higher concentration of 2 g/L where also the grilled meat flavor and saltiness were significantly enhanced.

Interestingly when the ribose conjugate of ornithine, i.e. Rib-Orn, was added to the chicken soup base at 0.25 g/L, both baked bread flavor and umami taste were significantly enhanced in comparison to the reference soup base.

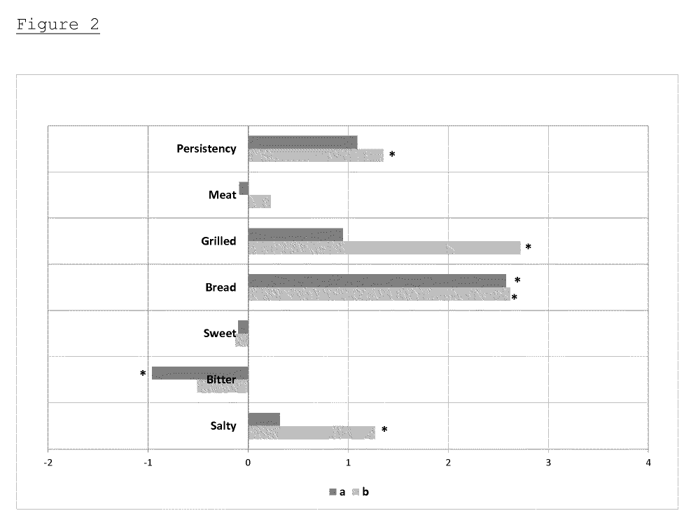

In conclusion, the addition of the sugar conjugate of ornithine modulates the flavor profile of the chicken soup and especially increases the desired grilled, bread flavors as well as saltiness and umaminess. The results are shown in FIG. 2.

### Summary of Sensory Results:

The table 2 summarizes the key sensory effects of the tested sugar conjugates.

TABLE 2

| | | Flavour | |
|---|---|---|---|
| | Known | property in | Sensory inpact on |
| Compounds | attributes | water (2 g/L) | chicken soup |
| Fru-Orn | | salty | grilled/popcorn, |
| | | | bread, saltiness |
| Fru-Orn-βala | | sweet, bread | grilled/popcorn, |
| | | | bread, saltiness |
| Orn-ala | Saltiness | astringent | none |
| | enhancing | | |
| | effect | | |
| Orn | Sweet | Not tested | Not tested |
| Rib-Orn | | Astringent, | Grilled/bread, |
| | | slightly sweet | umami |

### Example 6: Comparison Between the Soup Bases Containing the Sugar Conjugate 1-Deoxy-D-fructosyl-N-ornithine (Fru-orn) and the Mixture of Glucose and Ornithine

A first soup was prepared by adding 2 g/L (6.82 mmol/L) 1-deoxy-D-fructosyl-N-ornithine in the soup base described above. A second soup was prepared by adding same molar concentration of glucose and ornithine. The solutions were then evaluated by 6 panelists following the same procedure than described above. Obvious differences were found between the two samples: the soup containing 1-deoxy-D-fructosyl-N-ornithine was found more salty when tasted with nose-clip and with a greater chicken flavor when tasted without nose-clip.

### Example 7: Seasoning Compositions

Tomato soups can be prepared by dissolving 6 g tomato base powder as can be obtained in the commerce in 500 mL hot water. 1-deoxy-D-fructosyl-N-ornithine or alternatively 1-Deoxy-D-fructosyl-N-ornithyl-β-alanine can be added at a concentration of 2 g/L to the soups in order to improve their taste and flavor profile. The soups will then have a more pronounced grilled savory flavor as well as being perceived as slightly more salty than the corresponding reference soups without the addition of the ornithine comprising compounds.

1. Compound of the general formula I, 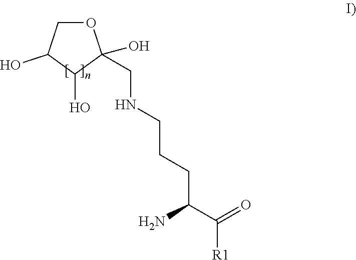
wherein R1 is hydroxyl (—OH) or an acid comprising an amino group, and wherein n is equal 1 or 2; or a salt of the compound.

2. The compound according to claim 1, wherein the acid comprising the amino group is selected from the group consisting of alanine, taurine, aspartic acid and glutamic acid.

3. The compound according to claim 1, wherein the compound with n equal 1 is a derivative of xylose or ribose.

4. The compound according to claim 1, wherein the compound with n equal 2 is a derivative of glucose.

5. The compound according to claim 1, which is selected from the group consisting of 1-deoxy-D-fructosyl-N-ornithine, 1-deoxy-D-fructosyl-N-ornithyl-β-alanine, 1-deoxy-2-pentulofuranos-1-yl-ornithine, and 1-deoxy-2-pentulofuranos-1-yl-ornithyl-β-alanine.

6. A composition comprising the compound of the general formula I, 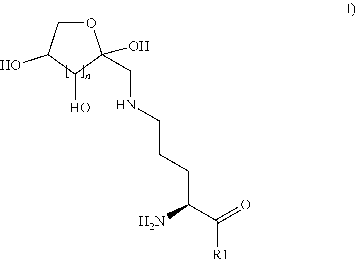
wherein R1 is hydroxyl (—OH) or an acid comprising an amino group, and wherein n is equal 1 or 2; or a salt of the compound in an amount of at least 0.25 mg/g.

7. The composition according to claim 6, wherein the composition is food grade.

8. The composition according to claim 7, wherein the composition is selected from the group consisting of a culinary seasoning product, a cooking aid, a sauce or soup concentrate, a dry and a wet pet-food product.

9-12. (canceled)

13. Method for enhancing the grilled meat and/or bread flavor of a culinary food product, comprising the step of adding the compound of the general formula I, 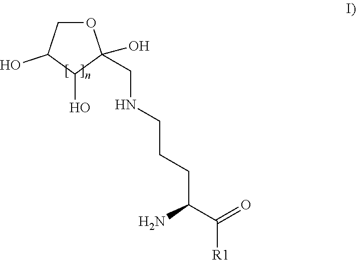
wherein R1 is hydroxyl (—OH) or an acid comprising an amino group, and wherein n is equal 1 or 2; or a salt of the compound to a food product.

14. (canceled)
